(19) United States
(12) Patent Application Publication
(10) Pub. No.: US 2005/0222675 A1
(43) Pub. Date: Oct. 6, 2005

(54) Implantable prosthetic heart valve comprising a valve body and a tubular vascular graft

(76) Inventor: Joseph A. Sauter, Austin, TX (US)

Correspondence Address: WILLIAMS, MORGAN & AMERSON, P.C., 10333 RICHMOND, SUITE 1100, HOUSTON, TX, 77042, US

(21) Appl. No.: 10/818,846

(22) Filed: Apr. 6, 2004

(51) Int. Cl. A61F 2/24

(52) U.S. Cl. 623/1.26; 623/2.4

(57) ABSTRACT

We disclose an implantable prosthetic heart valve, comprising: a valve body comprising an orifice member, wherein the orifice member comprises an external groove; a sewing cuff; a sewing cuff retaining member seated within the external groove of the orifice member and coupling the sewing cuff to the orifice member; a tubular vascular graft; and a graft retaining member seated within the external groove of the orifice member and coupling the tubular vascular graft to the orifice member.

### BACKGROUND OF THE INVENTION

The present invention relates generally to the field of prosthetic heart valves. More particularly, it concerns prosthetic heart valves combined with a tubular vascular graft.

In the mammalian heart, deoxygenated blood flows into the right atrium through the superior vena cava and the inferior vena cava. Upon contraction of the right atrium, the deoxygenated blood flows into the right ventricle. When the right ventricle contracts, the deoxygenated blood is pumped through the pulmonary artery to the lungs. Oxygenated blood returning from the lungs enters the left atrium. From the left atrium, the oxygenated blood flows into the left ventricle, which in turn pumps oxygenated blood to the body via the aorta and lesser arteries branching thereoff.

This pumping action is repeated in a rhythmic cardiac cycle in which the ventricular chambers alternately contract and pump, then relax and fill. As is well known, a series of one-way cardiac valves prevent backflow of the blood as it moves through the heart and the circulatory system. Between the atrial and ventricular chambers in the right and left sides of the heart are the tricuspid valve and the mitral valve, respectively. At the exits of the right and left ventricles are the pulmonic and aortic valves, respectively.

It is well known that various heart diseases may result in disorders of the cardiac valves. For example, diseases such as rheumatic fever can cause the shrinking or pulling apart of the valve orifice, while other diseases may result in endocarditis, an inflammation of the endocardium (membrane lining the heart). Resulting defects in the valves hinder the normal functioning of the atrioventricular orifices and operation of the heart. More specifically, defects such as the narrowing of the valve opening (valvular stenosis) or the defective closing of the valve (valvular insufficiency) result in an accumulation of blood in a heart cavity or regurgitation of blood past the valve. If uncorrected, prolonged valvular stenosis or valvular insufficiency can cause damage to the heart muscle, which may eventually necessitate total valve replacement.

These defects may be associated with any of the cardiac valves, although they occur most commonly in the left side of the heart. For example, if the aortic valve between the left ventricle and the aorta narrows, blood will accumulate in the left ventricle. Similarly, in the case of aortic valve insufficiency, the aortic valve does not close completely, and blood in the aorta flows back past the closed aortic valve and into the left ventricle when the ventricle relaxes.

In many cases, complete valve replacement is required. Mechanical artificial heart valves for humans are frequently fabricated from titanium, pyrolitic carbon, or biologic tissue, including tissue from cattle, swine, or man. Such valves have become widely accepted and used by many surgeons.

Mechanical prosthetic heart valves typically comprise a rigid orifice supporting one, two or three rigid occluders, or leaflets. The occluders pivot between open and shut positions and thereby control the flow of blood through the valve. The orifice and occluders are commonly formed of pyrolytic carbon, which is a particularly hard and wear-resistant form of carbon. To minimize deflection of the orifice and possible interference with the movement of the occluders, the orifice is often surrounded by a stiffening ring, which may be made of titanium, cobalt chromium, or stainless steel. In one valve configuration, the orifice and stiffening ring are captured within a knit fabric sewing or suture cuff. This prosthetic valve is placed into the valve opening and the sewing cuff is sutured to the patient's tissue. Over time, tissue grows into the fabric of the cuff, providing a secure seal for the prosthetic valve.

However, in many patients, once degeneration of a valve has occurred, it may occur that surrounding blood vessels are also diseased. Particularly in the case of the aortic valve, surgeons have found that the portion of the aorta adjacent to the valve is often degenerated to the degree that it must be replaced. Consequently, both the aortic valve and a segment of the ascending aorta may be replaced at the same time. When this technique was being developed, the surgeon would stitch a segment of vascular graft to the sewing ring of the mechanical valve after implanting the mechanical heart valve. However, this required a relatively long duration of surgery, and the quality of stitching could only be tested in vivo after implantation, making leaks difficult to detect and potentially deleterious to the well-being of the patient.

Subsequently, a valve having a preattached graft was developed. The graft is typically attached to the sewing ring. A drawback of this configuration is that the valve size has to be reduced in order to accommodate the additional bulk of the graft end. Hence, the valve implanted with this combination is generally smaller than that which a surgeon would ordinarily implant. This results in a restriction in the available flow area, with associated resistance to flow. Furthermore, the orifice area (pressure drop across the valve) is proportional to the fourth order power of the internal diameter of the valve. Thus, any decrease in the internal diameter of the valve is undesirable, as it reduces the volume of blood that can be pumped with the available heart muscle.

### SUMMARY OF THE INVENTION

In one embodiment, the present invention relates to an implantable prosthetic heart valve, comprising a valve body comprising an orifice member, wherein the orifice member comprises at least one external groove; a tubular vascular graft; and a graft retaining member seated within the external groove of the orifice member and coupling the tubular vascular graft to the orifice member.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a cross-sectional view of an implantable prosthetic heart valve according to one embodiment of the present invention.

FIG. 2 shows a cross-sectional view of an implantable prosthetic heart valve according to one embodiment of the present invention.

FIG. 3 shows a cross-sectional view of an implantable prosthetic heart valve according to one embodiment of the present invention.

FIG. 4 shows a cross-sectional view of an implantable prosthetic heart valve according to one embodiment of the present invention.

FIG. 5 shows a cross-sectional view of an implantable prosthetic heart valve according to one embodiment of the present invention.

FIG. 6 shows a cross-sectional view of an implantable prosthetic heart valve according to one embodiment of the present invention.

FIG. 7 shows a cross-sectional view of an implantable prosthetic heart valve according to one embodiment of the present invention.

FIG. 8 shows a cross-sectional view of an implantable prosthetic heart valve according to one embodiment of the present invention.

FIG. 9 shows a cross-sectional view of an implantable prosthetic heart valve according to one embodiment of the present invention.

### DESCRIPTION OF ILLUSTRATIVE EMBODIMENTS

The present invention will now be described with reference to the attached figures. The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than that understood by skilled artisans, such a special definition will be expressly set forth in the specification in a definitional manner that directly and unequivocally provides the special definition for the term or phrase.

The present invention relates to an implantable prosthetic heart valve, suitable for replacing a heart valve present in a valve annulus.

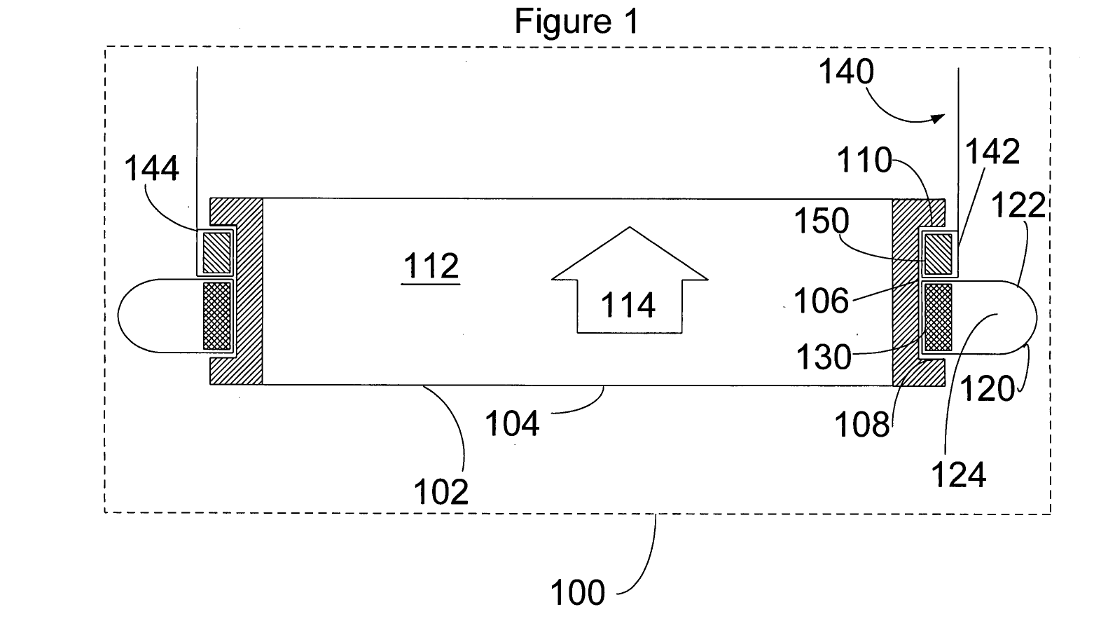

One embodiment of an implantable prosthetic heart valve according to the present invention is shown in cross-section in FIG. 1. The implantable prosthetic heart valve 100 comprises a means for defining a blood flow path, such as a valve body 102 comprising an orifice member 104, defining a blood flow annulus 112, or the like. (The implantable prosthetic heart valve 100 also comprises one or more leaflets, not shown, coupled to the valve body 102 by any coupling means known in the art, capable of reversibly closing the blood flow annulus 112). The orifice member 104 and the leaflets, not shown, may be made from any appropriate material, such as pyrolitic carbon, a polymer comprising carbon, a polymer comprising silicon, or others known in the art. The orifice member 104 may be toroidal or approximately toroidal in shape. The orifice member 104 comprises at least one external groove 106. In one embodiment, the external groove 106 is circumferential, i.e., extends around the entire circumference of the orifice member 104. In one embodiment, the external groove 106 may have an upstream shoulder 108 and a downstream shoulder 110. The implantable prosthetic heart valve 100 is designed for blood flow in the direction 114, i.e., in a direction approximately parallel to a line segment considered between the upstream shoulder 108 and the downstream shoulder 110.

In an exemplary embodiment, the external groove 106 has a width (e.g., a distance between the upstream shoulder 108 and the downstream shoulder 110) of from about 2.0 mm to about 8.0 mm, and a depth (e.g., the height of the upstream shoulder 108 or the downstream shoulder 110) of from about 0.2 mm to about 0.8 mm.

The implantable prosthetic heart valve 100 comprises means for attaching the implantable prosthetic heart valve 100 to the interior wall of a blood vessel. An example of such an attaching means is schematically depicted in FIGS. 1-9 as a sewing cuff 120 disposed about at least a portion, and possibly the entirety of, orifice member 104. The sewing cuff 120 may comprise one or more layers or folds of cloth 122 and, optionally, a filler 124, such as texturized yarn, polytetrafluoroethylene (Teflon®) felt, or molded silicon, among others known in the art. The sewing cuff 120 may comprise a suture lip or other portion suitable for affixing to tissue via suturing. An exemplary sewing cuff is described in U.S. Pat. No. 6,299,638, hereby incorporated herein by reference.

The sewing cuff 120 is coupled to the orifice member 104 by a coupling means, such as a sewing cuff retaining member 130 seated within an external groove 106 of the orifice member 104. In one illustrative embodiment, the sewing cuff retaining member 130 may be a solid ring, such as a split ring or the like. The sewing cuff retaining member 130 may be fabricated from cobalt-chromium alloy, stainless steel, or other biocompatible material. The sewing cuff retaining member 130 may be fabricated as a component of the sewing cuff 120, or the sewing cuff 120 can be attached to the sewing cuff retaining member 130, such as by stitching, after seating the sewing cuff retaining member 130 within an external groove 106.

In one exemplary embodiment, the sewing cuff retaining member 130 is a stainless steel wire from about 20 AWG to about 40 AWG (about 0.8 mm diameter to about 0.08 mm diameter).

The implantable prosthetic heart valve 100 also comprises a means for substituting for a diseased arterial segment, such as a tubular vascular graft 140, which may have a proximal graft end 142. (“Proximal,” in this context, refers to the end nearest the upstream shoulder 108). The tubular vascular graft 140 may be prepared according to techniques known in the art. In one embodiment, the tubular vascular graft 140 is cloth, such as woven polyethylene terephthalate (PET). In another embodiment, the tubular vascular graft 140 is a sinus valsalva taken from a mammalian donor, such as cattle, swine, or man.

The tubular vascular graft 140 is coupled to the orifice member 104 by coupling means, such as a graft retaining member 150 seated within the external groove 106 of the orifice member 104. In one illustrative embodiment, the graft retaining member 150 may be a solid ring, such as a split ring or the like. The graft retaining member 150 may be fabricated from cobalt-chromium alloy, stainless steel, or other biocompatible material. The graft retaining member 150 may be fabricated as a component of the tubular vascular graft 140, or the tubular vascular graft 140 may be attached to the graft retaining member 150, such as by stitching, after seating the graft retaining member 150 within the external groove 106. Typically, and in the embodiments shown in FIGS. 1-9, the tubular vascular graft 140 comprises a cloth layer which is wrapped around the graft retaining member 150. The cloth layer is typically stitched to itself at point 144 and excess cloth trimmed.

In one exemplary embodiment, the graft retaining member 150 is a stainless steel wire from about 20 AWG to about 40 AWG (about 0.8 mm diameter to about 0.08 mm diameter).

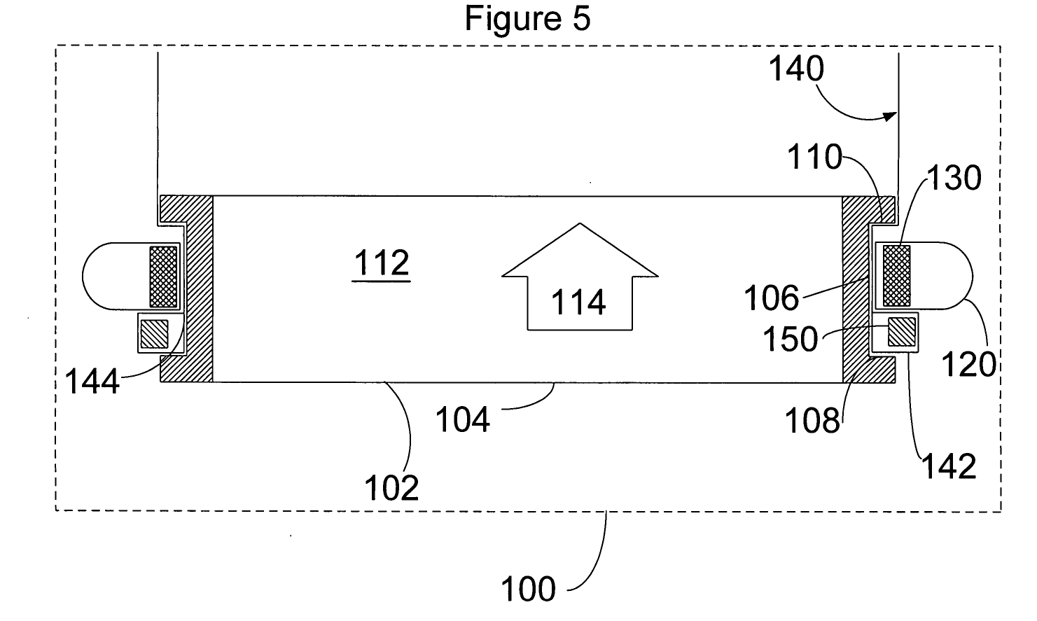

As will be apparent to the skilled artisan, there are two possible arrangements of the sewing cuff retaining member 130 and the graft retaining member 150. In one embodiment, shown in FIGS. 1-4, the graft retaining member 150 is seated between the sewing cuff retaining member 130 and the downstream shoulder 110 of the external groove 106. In another embodiment, shown in FIGS. 5-8, the graft retaining member 150 is seated between the sewing cuff retaining member 130 and the upstream shoulder 108 of the external groove 106.

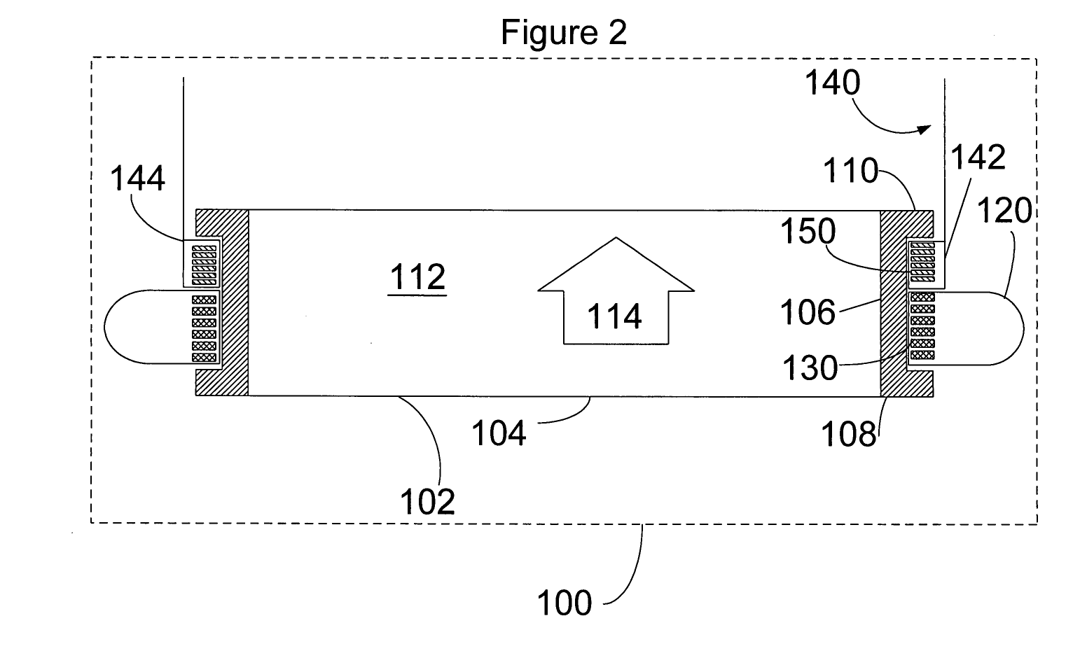

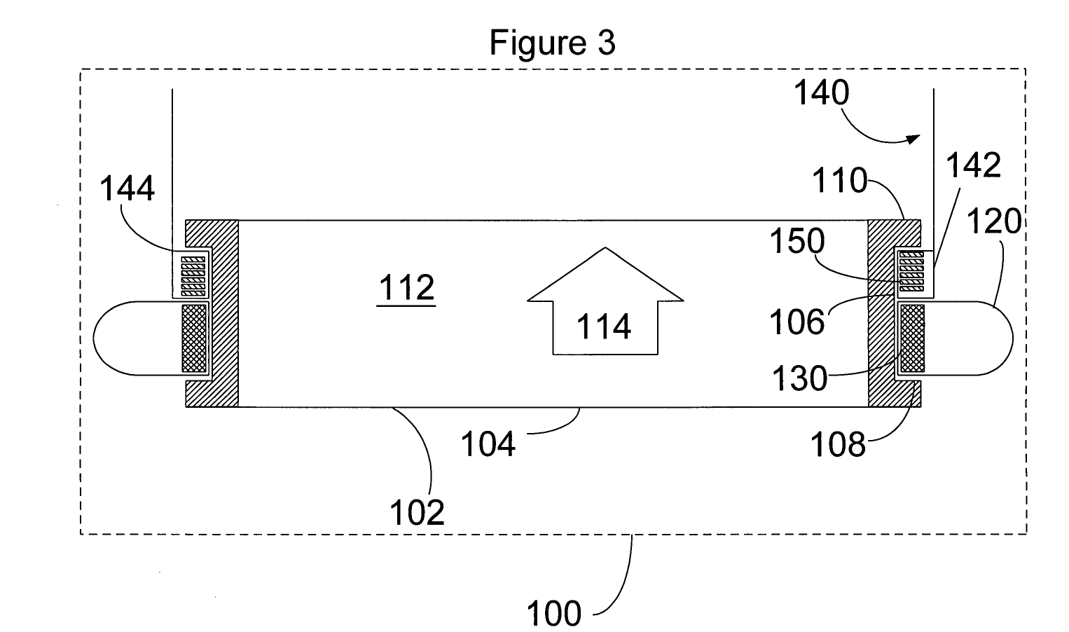

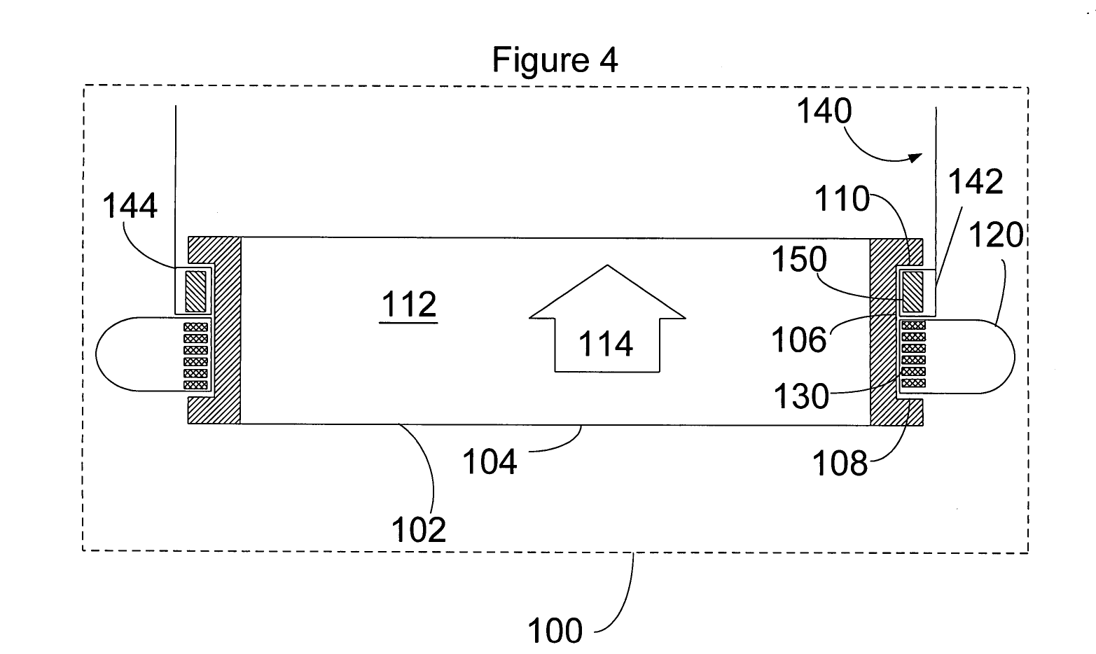

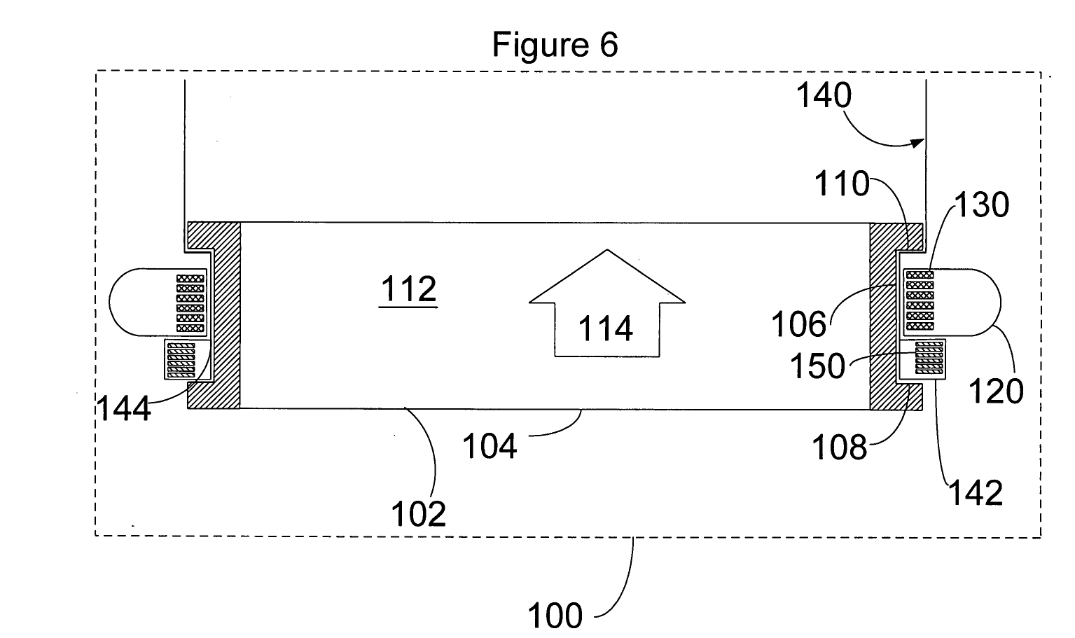

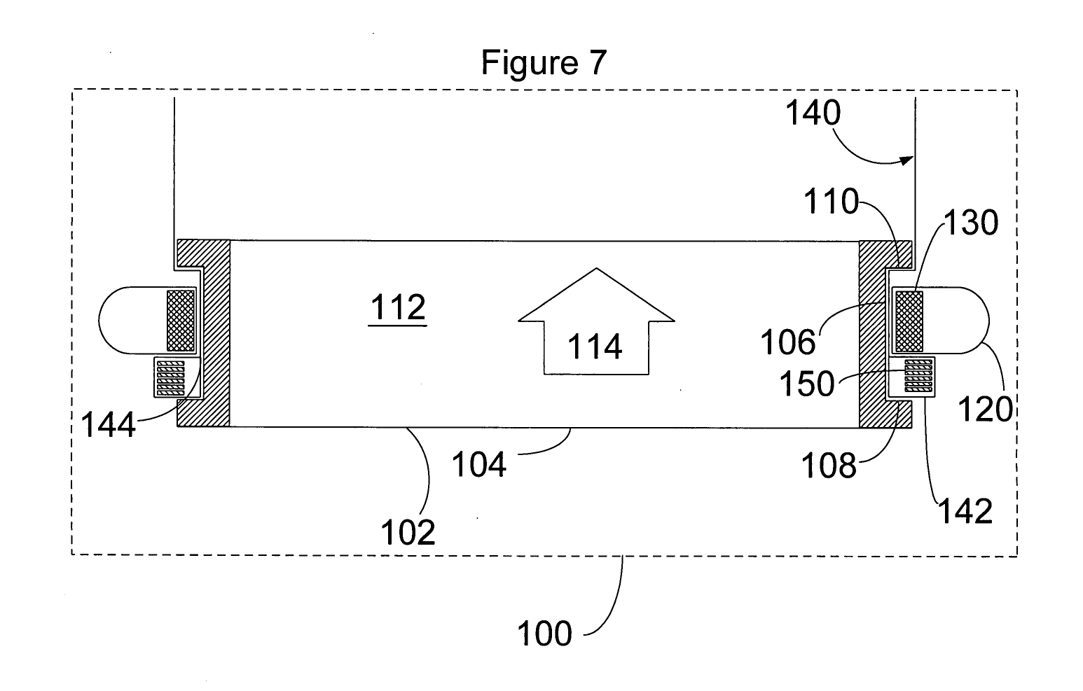

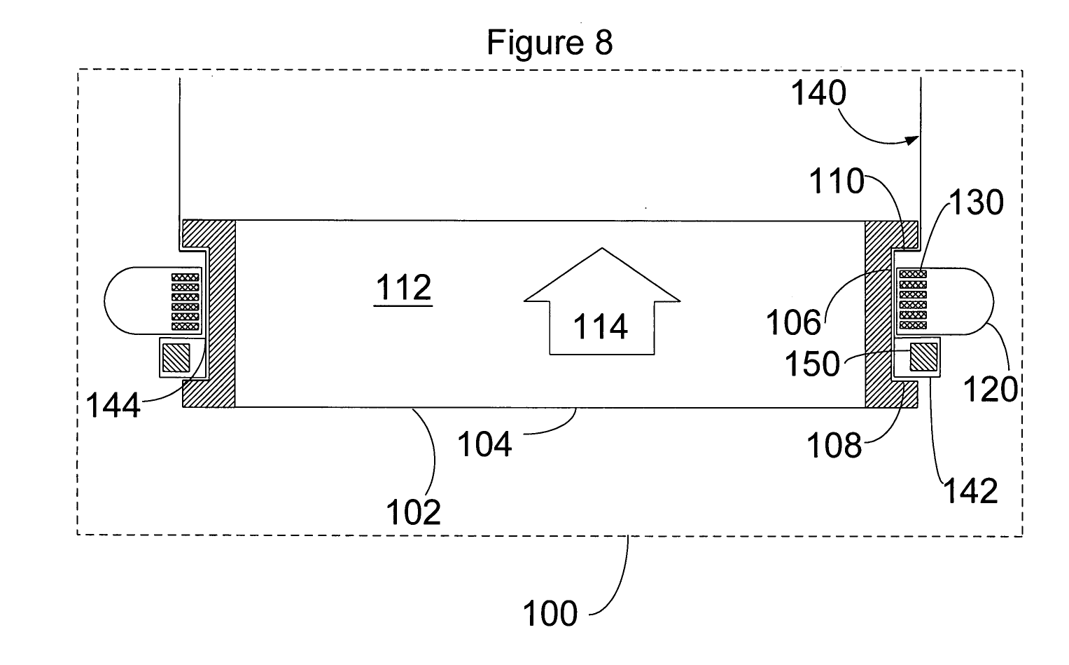

Instead of being a solid member, as shown in FIGS. 1 and 5, the sewing cuff retaining member 130, the graft retaining member 150, or both may be a helical ring, such as a spring. FIGS. 2 and 6 show an implantable prosthetic heart valve 100, wherein both the sewing cuff retaining member 130 and the graft retaining member 150 are springs. FIGS. 3 and 7 show an implantable prosthetic heart valve 100, wherein the sewing cuff retaining member 130 is a solid ring and the graft retaining member 150 is a spring. FIGS. 4 and 8 show an implantable prosthetic heart valve 100, wherein the sewing cuff retaining member 130 is a spring and the graft retaining member 150 is a solid ring.

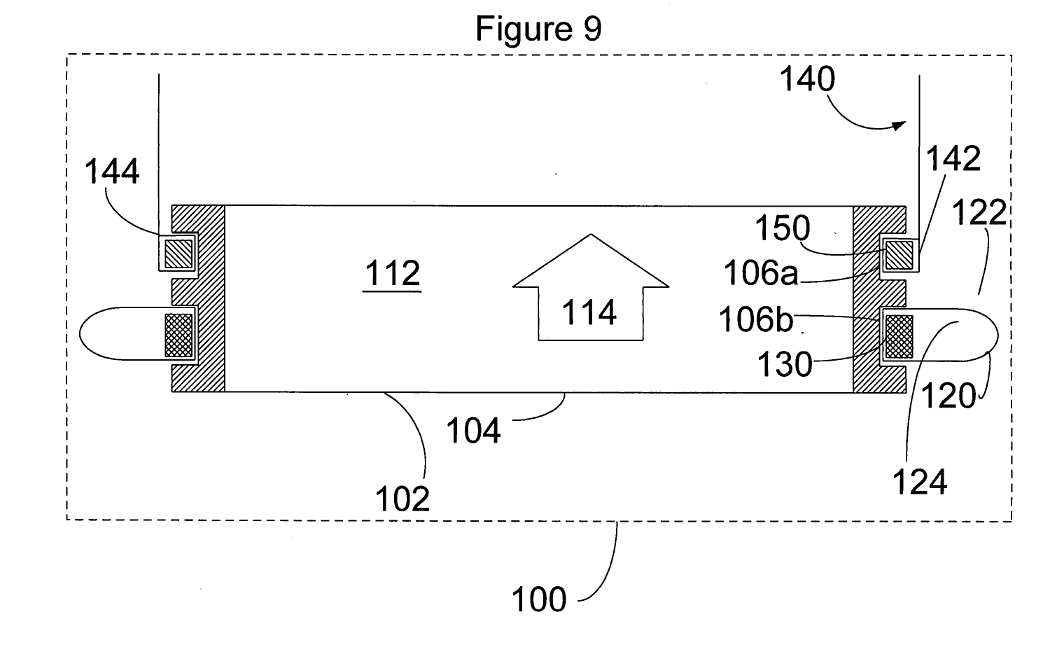

In the embodiments described above, the valve body 102 has a single external groove 106, in which both the sewing cuff retaining member 130 and the graft retaining member 150 are seated. An alternative embodiment is shown in FIG. 9, in which the valve body 102 has a first external groove 106a and a second external groove 106b, wherein the graft retaining member 150 (in FIG. 9, a solid ring) is seated in the first external groove 106a and the sewing cuff retaining member 130 (in FIG. 9, a solid ring) is seated in the second external groove 106b. (“First” and “second” are terms of convenience, and do not imply any particular order of the two external grooves in time or space). The skilled artisan will recognize that embodiments in which the valve body 102 has a first external groove 106a and a second external groove 106b, wherein the relative seating locations and types of the sewing cuff retaining member 130 and the graft retaining member 150 correspond to those of FIGS. 2-8, are within the scope of the present invention, though not shown.

A surgeon may use the implantable prosthetic heart valve 100 to replace a defective native, natural, or prosthetic heart valve and an adjacent defective native or natural vascular segment, according to techniques known in the art. In one embodiment, the present invention relates to a method of implanting a prosthetic heart valve comprising a tubular vascular graft into a patient, comprising removing a prior heart valve and adjacent arterial section from the vasculature of the patient; placing the prosthetic heart valve into the position formerly occupied by the removed prior heart valve, wherein the prosthetic heart valve comprises a valve body comprising an orifice member, wherein the orifice member comprises an external groove; a sewing cuff; a sewing cuff retaining member seated within the external groove of the orifice member and coupling the sewing cuff to the orifice member; a tubular vascular graft; and a graft retaining member seated within the external groove of the orifice member and coupling the tubular vascular graft to the orifice member; and attaching the prosthetic heart valve and tubular vascular graft to the vasculature of the patient. The attachment is typically performed between the prosthetic heart valve and tubular vascular graft and a fibrous ring of annular tissue in the vascular system of the patient. In one embodiment, attaching may be effected by stitching between the sewing cuff and the annular tissue of the patient, and by stitching between the tubular vascular graft and the annular tissue of the patient.

In one embodiment, the defective heart valve is the aortic valve, and the defective vascular segment is the sinus valsalva of the aorta.

In another embodiment, the present invention relates to a method of attaching a tubular vascular graft to a valve body comprising an orifice member, wherein the orifice member comprises at least one external groove, the method comprising:
coupling the tubular vascular graft to the orifice member with a graft retaining member seated within the external groove of the orifice member.

Coupling may be effected in a number of ways. In one embodiment, the graft retaining member is a split ring that is seated in the external groove; the split is then substantially closed by mechanical actuation; and the tubular vascular graft is then affixed to the seated graft retaining member by stitching. Alternatively, the graft retaining member can be sufficiently pliant to deflect around non-groove portions of the orifice member and subsequently be seated in the external groove, with subsequent affixture of the tubular vascular graft to the seated graft retaining member by stitching. In other embodiments, the tubular vascular graft is at least partially affixed to the graft retaining member, such as by stitching, and then the graft retaining member is seated in the external groove of the orifice member.

All of the apparatus disclosed and claimed herein may be made and executed without undue experimentation in light of the present disclosure. While the compositions and methods of this invention have been described in terms of preferred embodiments, it will be apparent to those of skill in the art that variations may be applied to the apparatus described herein without departing from the concept, spirit and scope of the invention.

1. An implantable prosthetic heart valve, comprising:
a valve body comprising an orifice member, wherein the orifice member comprises a least one external groove;
a tubular vascular graft; and
a graft retaining member seated within the external groove of the orifice member and coupling the tubular vascular graft to the orifice member.

2. The implantable prosthetic heart valve of claim 1, further comprising a sewing cuff, and a sewing cuff retaining member seated within an external groove of the orifice member and coupling the sewing cuff to the orifice member.

3. The implantable prosthetic heart valve of claim 1, wherein the sewing cuff retaining member is a solid ring.

4. The implantable prosthetic heart valve of claim 1, wherein the sewing cuff retaining member is a spring.

5. The implantable prosthetic heart valve of claim 1, wherein the graft retaining member is a solid ring.

6. The implantable prosthetic heart valve of claim 1, wherein the graft retaining member is a spring.

7. The implantable prosthetic heart valve of claim 1, comprising one external groove, wherein the external groove has an upstream shoulder and a downstream shoulder and the graft retaining member is seated between the sewing cuff retaining member and the upstream shoulder of the external groove.

8. The implantable prosthetic heart valve of claim 1, comprising one external groove, wherein the external groove has an upstream shoulder and a downstream shoulder and the graft retaining member is seated between the sewing cuff retaining member and the downstream shoulder of the external groove.

9. The implantable prosthetic heart valve of claim 1, comprising two external grooves, wherein graft retaining member is seated in a first external groove and the sewing cuff retaining member is seated in a second external groove.

10. An implantable prosthetic heart valve, comprising:
a means for defining a blood flow path;
a means for substituting for a diseased vascular segment; and
a means for coupling the tubular vascular graft to the means for defining a blood flow path.

11. The implantable prosthetic heart valve of claim 10, further comprising a means for attaching the implantable prosthetic heart valve to the interior wall of a blood vessel, and a means for coupling the attaching means to the means for defining a blood flow path.

12. A method of attaching a tubular vascular graft to a valve body comprising an orifice member, wherein the orifice member comprises at least one external groove, the method comprising:
coupling the tubular vascular graft to the orifice member with a graft retaining member seated within the external groove of the orifice member.

13. A method of implanting a prosthetic heart valve comprising a tubular vascular graft into a patient, comprising:
removing a prior heart valve and adjacent arterial section from the vasculature of the patient;
placing the prosthetic heart valve into the position formerly occupied by the removed prior heart valve, wherein the prosthetic heart valve comprises a valve body comprising an orifice member, wherein the orifice member comprises at least one external groove; a sewing cuff; a sewing cuff retaining member seated within an external groove of the orifice member and coupling the sewing cuff to the orifice member; a tubular vascular graft; and a graft retaining member seated within an external groove of the orifice member and coupling the tubular vascular graft to the orifice member; and
attaching the prosthetic heart valve and tubular vascular graft to the vasculature of the patient.

14. The method of claim 13, wherein the position formerly occupied by the removed prior heart valve is the aortic valve position, and the adjacent arterial section is the sinus valsalva.
